(19) United States
(12) Patent Application Publication
(10) Pub. No.: US 2025/0331733 A1
(43) Pub. Date: Oct. 30, 2025

(54) COMPENSATION OF IMPEDANCE-BASE ELECTRODE POSITIONS

(71) Applicant: St. Jude Medical, Cardiology Division, Inc., St. Paul, MN (US)

(72) Inventors: Hana Boudlali, Blaine, MN (US); Hai Huang, Woodbury, MN (US)

(21) Appl. No.: 19/191,008

(22) Filed: Apr. 28, 2025

Related U.S. Application Data
(60) Provisional application No. 63/640,316, filed on Apr. 30, 2024.

(51) Int. Cl. A61B 5/06; A61B 5/00

(52) U.S. Cl. CPC A61B 5/068; A61B 5/6852; A61B 5/742; A61B 2560/0468; A61B 2562/0223

(57) ABSTRACT

A method of displaying a position of a variable loop catheter that includes a distal feature having a plurality of electrodes located along a length of the distal feature. Raw impedance-based positions of each of the plurality of electrodes located at a distal end of the variable loop catheter are calculated based on voltages sensed by each of the plurality of electrodes. The method includes determining whether a loop is formed by the distal feature based on the raw impedance-based positions of each of the plurality of electrodes. A radius of the loop formed by the distal feature is calculated based on a detected overlap between respective electrodes. The measured raw impedance-based positions of each of the plurality of electrodes is corrected based on the calculated radius of the loop.

### BACKGROUND

The present disclosure relates generally to systems and methods for localization and visualization of a catheter.

Various systems are known for determining the position and orientation (P&O) of a medical device in a human body, for example, for visualization and navigation purposes. One such system is known as an impedance-based localization system. Impedance-based systems generally include one or more pairs of body surface electrodes (e.g., patches) outside a patient's body, a reference sensor (e.g., another patch) attached to the patient's body, and one or more sensors (e.g., electrodes) located on the medical device. In general, impedance-based localization includes applying a current across pairs of body surface electrodes that creates a voltage gradient between the body surface electrodes (sometimes also referred to as an impedance field or gradient as the voltage gradient depends on the impedance of the tissue). The electrodes located on the medical device sense a voltage, wherein the sense voltage can be compared to the voltage gradient to determine the location of the electrode within the patient's body.

However, the voltage gradient varies based on the impedance of the tissue, wherein inhomogeneities in tissue impedance may cause errors in the determined position of electrodes. Therefore, there exists a need to correct and/or verify accuracy of impedance-based positions.

### SUMMARY

A method of displaying a position of a variable loop catheter that includes a distal feature having a plurality of electrodes located along a length of the distal feature. Raw impedance-based positions of each of the plurality of electrodes located at a distal end of the variable loop catheter are calculated based on voltages sensed by each of the plurality of electrodes. The method includes determining whether a loop is formed by the distal feature based on the raw impedance-based positions of each of the plurality of electrodes. A radius of the loop formed by the distal feature is calculated based on a detected overlap between respective electrodes. The measured raw impedance-based positions of each of the plurality of electrodes is corrected based on the calculated radius of the loop.

A medical positioning system includes a variable loop catheter including an elongate shaft and a distal feature including a plurality of distal electrodes. The plurality of distal electrodes sense an impedance field. An electronic control unit (ECU) is in communication with the catheter. The ECU receiving the sensed impedance field at the plurality of distal electrodes to measure a raw impedance-based position of each of the plurality of distal electrodes. The ECU determines whether the distal feature forms a loop in an overlapping state based on the measured raw impedance-based positions of each of the plurality of distal electrodes. The ECU corrects the measured raw impedance-based positions of each of the plurality of distal electrodes based on detected characteristics of the loop in the overlapping state.

A method of correcting impedance-based electrode positions for electrodes on a distal feature of a catheter includes calculating raw impedance-based positions of each of a plurality of electrodes located at a distal end of a variable loop catheter based on voltages sensed by each of the plurality of electrodes. A longitudinal shaft axis between two or more shaft sensors is generated. A longitudinal range of deflection is determined based on the longitudinal shaft axis. The measured raw impedance-based positions of each of the plurality of electrodes is compared to the longitudinal range of deflection. The measured raw impedance-based positions of each of the plurality of electrodes is corrected in a longitudinal direction if the measured raw impedance-based positions are outside of the longitudinal range of deflection.

There is also provided a computer readable medium, a record carrier or a computer program product comprising instructions that, when executed, cause a computer or processor to perform any of the methods set forth herein.

These and other examples and features of the present devices, systems, and methods will be set forth, at least in part, in the following Detailed Description. This Overview is intended to provide non-limiting examples of the present subject matter-it is not intended to provide an exclusive or exhaustive explanation. The Detailed Description below is included to provide further information about the present devices, systems, and methods.

It will also be appreciated that the methods undertaken herein, including the various calculations and determinations, may be undertaken by a processor or a computer on data representative of the received signals, for example, the received voltages sensed by each of the plurality of electrodes.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 is a diagrammatic view of a medical positioning system and catheter, according to some embodiments.

FIG. 2 is an isometric front view of a loop catheter, according to some embodiments.

FIG. 3 is a visualization display of catheter position based on raw impedance measurements overlayed with a corrected visualization display, according to some embodiments.

FIG. 4 is a flow chart of a method of correcting impedance-based electrode positions for electrodes on a variable loop catheter, according to some embodiments.

FIG. 5 is a flow chart of a method of determining whether an overlapping loop is formed by the distal feature, according to some embodiments.

FIG. 6 is a flow chart of a method of calculating the radius of the overlapping loop with overlapping electrode data, according to some embodiments.

FIG. 7A is an exemplary coordinate system of electrode position measurements of a variable loop catheter, according to some embodiments.

FIG. 7B is an exemplary coordinate system of electrode position measurements of a variable loop catheter, according to some embodiments.

FIG. 7C is a top view of a simplified coordinate system of FIG. 7B, according to some embodiments.

FIG. 7D is an exemplary diagrammatic view of a cross product of two vectors, according to some embodiments.

FIG. 7E is a diagrammatic view of an exemplary elliptical catheter loop, according to some embodiments

FIG. 8A is a visualization display of catheter position/geometry based on raw impedance-based measurements, according to some embodiments.

FIG. 8B is a visualization display of corrected catheter position/geometry, according to some embodiments.

FIG. 9A is an isometric front view of a loop catheter, according to some embodiments.

FIG. 9B is an isometric front view of a loop catheter in a maximum inversion deflection state, according to some embodiments.

FIG. 9C is an isometric front view of a loop catheter in a maximum axial stretch deflection state, according to some embodiments.

FIG. 10 is a flow chart of a method of rectifying impedance-based electrode positions for electrodes on a distal feature of a catheter, according to some embodiments.

### DETAILED DESCRIPTION

According to some embodiments, this disclosure relates to devices, systems, and methods for determining the location of a medical device within a patient using an impedance-based localization system. A common source of error in impedance-based localization systems is due to inhomogeneous tissue impedances. In some embodiments, the geometry of the medical device can be utilized to correct errors in raw impedance-based locations. In some embodiments, the medical device includes a distal feature having a plurality of electrodes, wherein the distal feature is circular or lasso-shaped and has a variable radius. The method includes detecting overlapping electrode positions indicative of the loop radius. For example, if the proximal-most electrode on the distal feature overlaps any other electrode on the distal feature, an overlapping loop is formed (and vice-versa for the distal-most electrode). The radius of the variable radius loop may be calculated by determining which electrode pairs are overlapping and referencing stored data regarding the longitudinal lengths (i.e., the unraveled distances) between electrode pairs. The raw impedance-based locations of each of the plurality of electrodes is corrected based on the calculated radius of the overlapping loop.

A method of compensating for distortions in raw impedance-based electrode positions of a variable loop catheter utilizes the physical geometry and mechanical constraints of the variable loop catheter to determine whether a distortion has occurred. For example, the mechanical constraints of the variable loop catheter may limit the position of a first electrode relative to a second electrode, i.e., the distance between the first electrode and the second electrode is physically constrained within a narrow range. Thus, if raw impedance-based electrode positions indicate that the distance between the first electrode and the second electrode is outside of the possible range (i.e., outside of the physical constraints), the method may recognize a distortion has occurred and correct the raw impedance-based electrode positions to compensate for the distortion.

The catheter includes one or more shaft sensors disposed on or within the catheter shaft (proximal to the distal feature). The one or more shaft sensors measure a position and/or orientation. In some embodiments, the method includes generating a longitudinal shaft axis with the measured positions of the one or more shaft sensors. The mechanical constraints of the catheter and the distal feature limit travel of one or more electrodes positioned on the distal feature relative to the longitudinal shaft axis. The method includes compensating the detected position of the one or electrodes based on the comparison of the longitudinal shaft axis and the raw impedance-based locations of the one or electrodes.

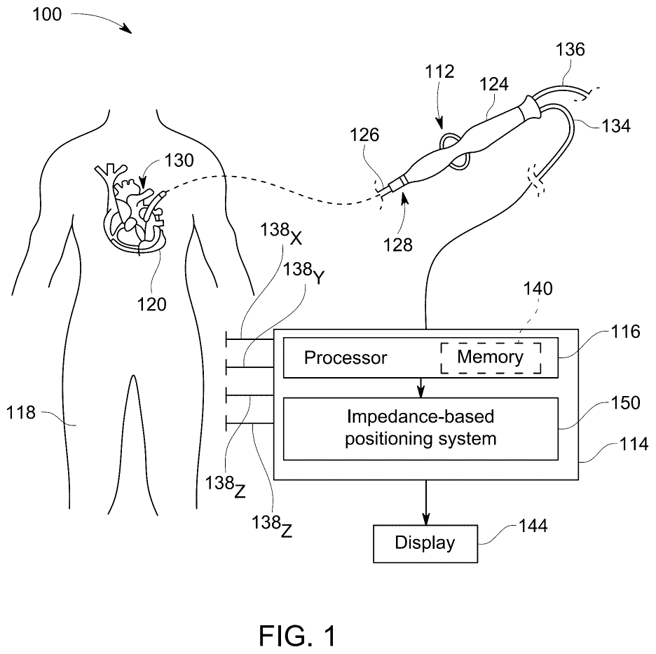

FIG. 1 is diagrammatic view of a medical positioning system 100 and catheter 112, according to some embodiments. The medical positioning system 100 includes a processing system 114 and an catheter 112. In some embodiments, the catheter 112 includes a proximal end 128, a shaft 126, and a distal end 130. A handle 124 is located at the proximal end 128 and allows a technician to guide/steer the distal end 130 of the catheter 112 within the patient's vasculature, including within a patient's heart 120. At least one electrode for mapping, ablation, and/or navigation/tracking may be provided at the distal end region of the catheter 112. The processing system 114 includes a processor 116 (e.g., electronic control unit (ECU)) with memory 140, an impedance-based positioning system 150, and a display device 144. In some embodiments, the processing system 114 includes a magnetic-based positioning system (not shown). The processing system 114 may be in electrical communication with the catheter 112, for example via first cable 134. A second cable 136 may be in electrical communication with other medical systems (e.g., generator for providing ablation therapy). The processing system 114 may also be in electrical communication with patch electrodes 138 attached to the body of the patient 118. It will be appreciated that the processing system 114 that receives electrical signals (from the catheter 112 or other medical systems) is configured to represent the electrical signals as data, such that processor 116 (e.g., electronic control unit (ECU)) can process the data, store the data temporarily or permanently in memory 140, use the data to drive display device 144 and provide data to and receive data from the impedance-based positioning system 150. The determinations, calculations and manipulations undertaken by processing system 114 in the foregoing are understood to be determinations, calculations and manipulations on data representative of the received electrical signals.

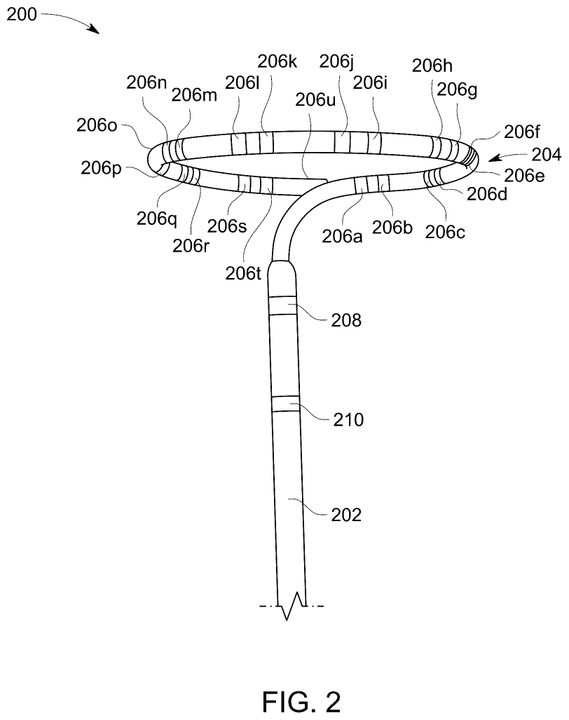

In some embodiments, processing system 114 implements impedance-based localization to determine the position of the one or more electrodes located at the distal end 130 of the catheter 112. The patch electrodes 138 may form a part of an impedance-based electrode location system. Pairs of patch electrodes 138_{X}, 138_{Y}, 138_{Z} are placed on the body along a first (x) axis, a second (y) axis, and a third (z) axis. Excitation of patch electrodes pairs 138_{X}, 138_{Y}, 138_{Z} generates orthogonal voltage gradients within an area of interest of the body, such as the heart 120. Patch electrode 138B may be attached to the stomach of the patient 118. Catheter 112 includes one or more electrodes (shown in FIG. 2) that are utilized to sense a voltage, wherein the sensed voltage corresponds with an impedance that can be utilized by the processing system to determine a location of the electrode within the patient's body 118—referred to herein as a raw impedance-based electrode location system. In some embodiments, the voltage sensed by each electrode is communicated to the processor system 114 via cable 134 for processing. In addition, in some embodiments the processing system 114 may further implement magnetic-based localization to determine the position and/or orientation of one or more magnetic sensors located at the distal end 130 of the catheter 112. For magnetic-based localization, a magnetic field is generated by an external magnetic field generator (not shown). In some embodiments, the catheter 112 includes one or more sensors configured to detect one or more of the magnetic fields, which may also be communicated to the processing system 114 via cables 134. The processing system 114 utilizes the measured magnetic fields to determine the location and/or orientation of one or more of the sensors-referred to herein as a magnetic-based location system.

In some embodiments, the impedance-based locations of the one or more electrodes-either alone or in combination with one or more magnetic-based locations-are utilized to generate a visual display or output presented by display device 144 that allows a operator to visualize the distal end 130 of the catheter 112 within the patient's body. However, as described in more detail below, inhomogeneity of the tissue causes errors in the raw impedance-based localization estimates, and may make it difficult for an operator to be able to visualize how the distal end 130 of the catheter 112 is positioned within the body. As described in more detail below, in some embodiments the geometry of the distal feature located at the distal end 130 of the catheter 112 can be utilized to correct the raw impedance-based localization estimates. For example, in embodiments in which the distal feature is a variable radius lasso or circular feature, the raw impedance-based localization estimates can be compared with one another to detect overlap between respective electrodes. That is, an electrode located at a distal end of the distal feature makes a full circle and overlaps with a more proximally located electrode. Detection of the overlapping electrodes provides information regarding the radius of the variable radius lasso or circular feature. Having determined the radius of the lasso or circular feature, this information can be utilized to correct the raw impedance-based localizations of each of the plurality of electrodes located on the lasso or circular feature.

FIG. 2 is an isometric front view of a variable loop catheter 200, according to some embodiments. The catheter 200 includes an elongate shaft 202 and a distal feature 204 having a plurality of electrodes 206a-206u. The plurality of electrodes 206 include a proximal electrode 206a (i.e., the proximal-most electrode on the distal feature 204) and a distal electrode 206u (i.e., the distal-most electrode on the distal feature 204). The plurality of electrodes 206 include electrodes 206b-206t along the distal feature 204. In some embodiments, each of the plurality of electrodes 206a-206u are evenly distributed on the distal feature 204. In other embodiments, each of the plurality of electrodes 206a-206u are distributed in pairs (i.e., electrodes 206a, 206b are positioned adjacent to each other, electrodes 206c, 206d are positioned adjacent to each other, etc.) on the distal feature 204. In some embodiments, the catheter 200 includes one or more shaft sensors, including for example, ring electrodes 208, 210. In some embodiments, the shaft sensors 208, 210 are electrodes configured to sense a voltage gradient generated by an impedance-based electrode location system. In other embodiments, the shaft sensors 208, 210 are magnetic sensors configured to sense a magnetic field generated by a magnetic-based location system.

In some embodiments, the distal feature 204 of the catheter 200 is a variable loop feature. The variable loop feature is configured to form a loop (i.e., a circular, semicircular, and/or elliptical shape) with an elongated flexible member. The size of the loop (radius) varies depending on the application force, contact surface, and/or physical properties of the distal feature 204. As the radius of the variable loop decreases, an overlap is formed by the distal feature, i.e., the distal end of the distal feature 204 overlaps the proximal end of the distal feature 204. Stated differently, the distal electrode 206u overlaps the proximal electrode 206a along the same circular/elliptical path. As described in more detail below, detecting overlap between particular electrodes provides information regarding the radius of the distal feature 204. For example, if electrode 206u overlaps with electrode 206a, a determination can be made regarding the radius of the loop based on the known length of the loop. If electrode 206u overlaps with electrode 206c, a determination can be made regarding the radius of the loop—in this case, the radius is known to be smaller than in the embodiment in which electrode 206u overlaps only with electrode 206a.

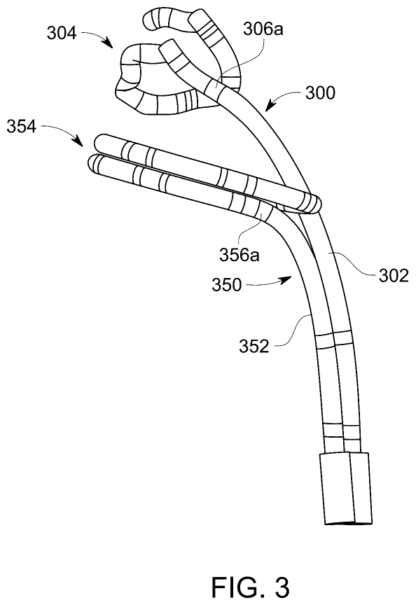

FIG. 3 is a visualization display of catheter position 300 generated based on raw impedance-based measurements overlayed with a corrected visualization display of catheter position 350 corrected according to embodiments described herein The visualization displays shown in FIG. 3 represents the type of display that may be presented to an operator via display 114 by processing system 114. The visualized raw-impedance based catheter position 300 includes a visualized shaft 302 and a visualized distal feature 304. The corrected visualized catheter 350 includes a corrected shaft 352 and a corrected distal feature 354.

The visualized distal feature 304 is an imperfect, irregular shape. In some embodiments, the raw impedance-based catheter position measurements are distorted by non-homogenous conductive properties of different biologic tissue within a patient's body. For example, muscular tissue, vascular tissue, bone, cartilage, blood, etc., have different conductive properties which could create an non-homogeneous impedance field. The non-homogeneous impedance field can distort the raw impedance-based catheter position measurements, resulting in an imperfect, irregular shape of the visualized distal feature 304.

The visualized distal feature 304 (as illustrated in FIG. 3) has a shape that is impossible for the distal feature 204 to form in real, three dimensional space. The mechanical or physical constraints of the distal feature 204 limit the maximum/minimum loop size formed by the distal feature, limit the relative positions of the plurality of electrodes 206a-206u, and/or limit the position of the distal feature 204 relative to the elongate shaft 202. In other words, the catheter 200 is unable to form the shape depicted by the visualized distal feature 304 without tearing, breaking, or damaging the distal feature 204. For example, the distance between the elongate shaft 202 and the proximal electrode 206a is limited, as the elongate shaft 202 and/or distal feature 204 cannot stretch past a maximum length. Thus, raw impedance-based position of the proximal electrode 206a (shown as visualized electrode 306a) is separated from the visualized shaft 302 by a distance exceeding the physical constraint of the catheter 200, a correction to the raw impedance-based position is appropriate.

The corrected visualized catheter 350 is an exemplary correction to the raw impedance-based catheter visualization 300. For instance, the raw impedance-based position of the proximal electrode 206a is corrected to a corrected visualized electrode (within the bounds of the physical constraint of the catheter). The shape formed by the distal feature 204 is corrected to form the corrected visualized feature 304. As discussed in more detail below, in some embodiments the overlapping of electrodes located on the distal feature 204 is utilized to determine the radius of the variable radius loop. Based on the known radius, the raw impedance measurements can be corrected to accommodate the known radius of the loop, providing a geometry that aligns with the physical constraints of the distal feature 204. As will be appreciated, the corrected visualization more accurately informs a user of the geometry of the distal feature 204 and which may aid subsequent navigation of the distal feature 204 within the patient's vasculature, or heart 120.

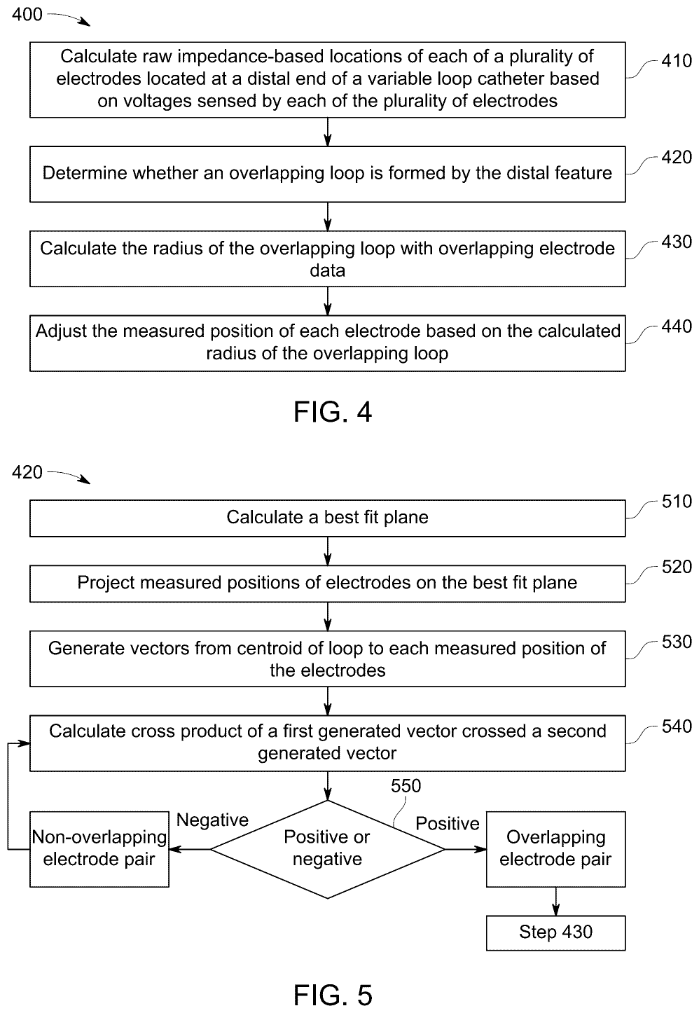

FIG. 4 is a flow chart of a method 400 of correcting the raw impedance-based electrode positions for electrodes on a variable loop catheter, according to some embodiments. At step 410, the method 400 includes calculating raw impedance-based locations of each of a plurality of electrodes 206a-206u located at a distal feature 204 of a variable loop catheter 200 based on signals sensed by each of the plurality of electrodes 206a-206u. For instance, a voltage gradient is generated within a body 118 of a patient by driving current through one or more pairs of patch electrodes 138_{X}, 138_{Y}, 138_{Z}, 138_{B}, secured to the body 118. Each of the plurality of electrodes 206a-206u senses the voltage gradient and communicates with a processing system 114. The processing system 114 calculates raw impedance-based locations of each of a plurality of electrodes 206a-206u located at a distal feature 204 based on the sensed voltage gradient at each of a plurality of electrodes 206a-206u, according to some embodiments.

At step 420, a determination is made as to whether an overlapping loop is formed by the distal feature 204 based on the raw impedance-based locations of each of the plurality of electrodes 206a-206u. For instance, if the proximal-most electrode 206a overlaps the distal-most electrode 206u (or vice-versa), then a determination is made that an overlapping loop is formed. In some embodiments, the method illustrated in FIG. 5 is used to determine whether an overlapping loop is formed by the distal feature 204 based on the raw-impedance based locations.

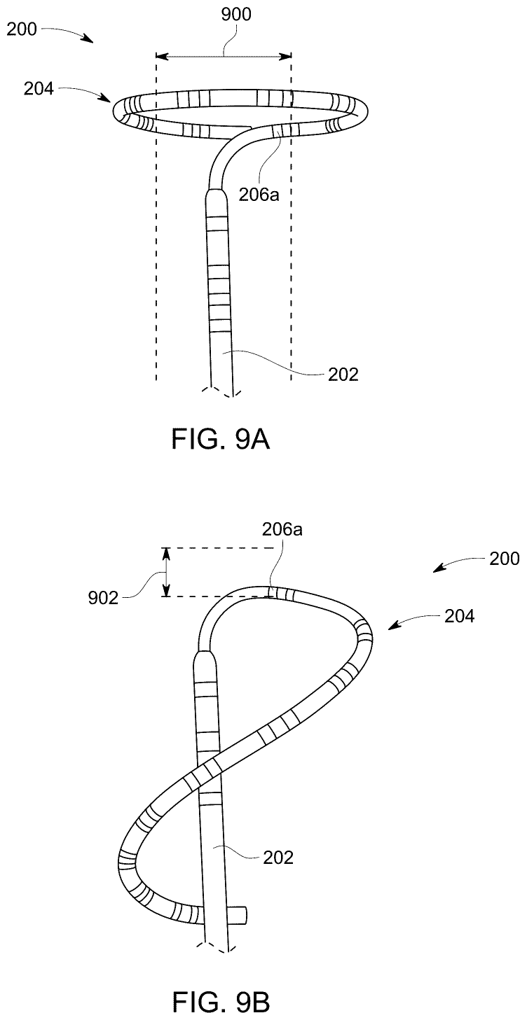

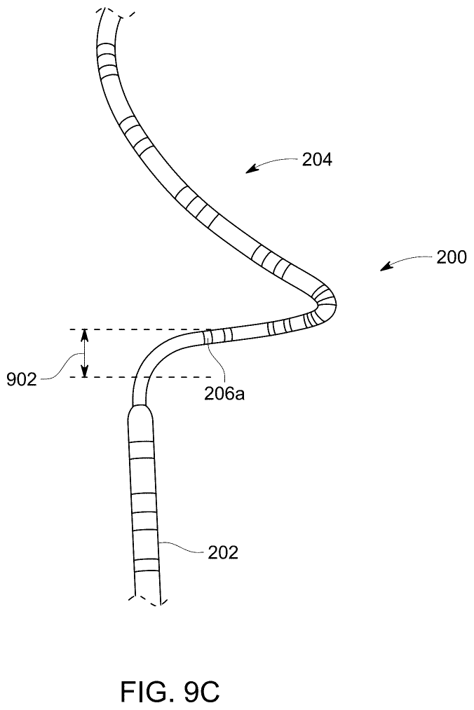

In some embodiments, the step 420 includes determining whether an irregular loop shape is formed, i.e., the distal feature 204 is caught against tissue in a way which uncoils or unravels the distal feature 204 (e.g., as shown in FIG. 9B and FIG. 9C). For instance, a residual is calculated based on the raw-impedance based locations of each of the plurality of electrodes 206a-206u. The residual is a difference in the axial direction between the measured raw-impedance based locations and a best fit plane of the distal feature 204. In some embodiments, determining whether an irregular loop shape is formed includes comparing the calculated residual to a threshold. If the calculated residual exceeds the threshold, a determination is made that an irregular loop shape is formed. In other words, if the measured raw-impedance based axial locations of the electrodes 206a-206u deviate from the best-fit plane past a threshold amount, a determination is made that the distal feature 204 is caught in an irregular loop shape.

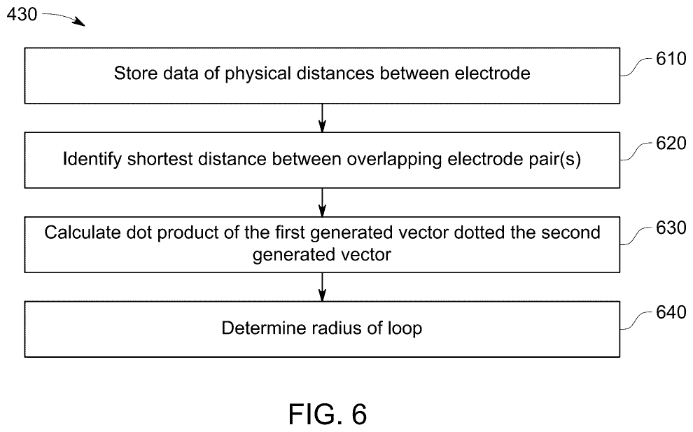

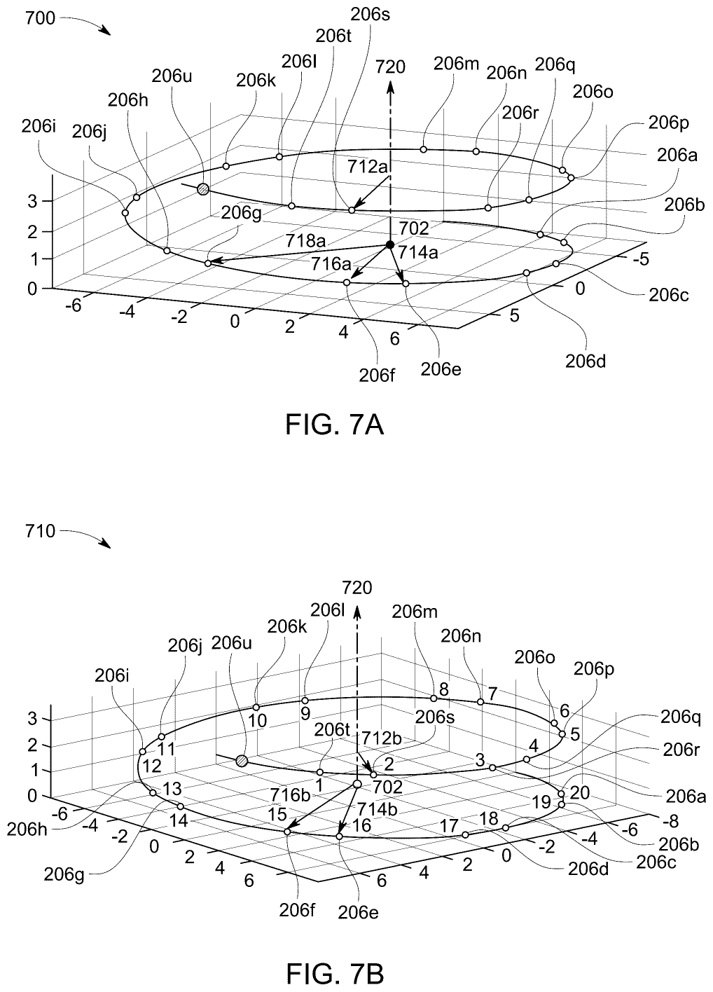

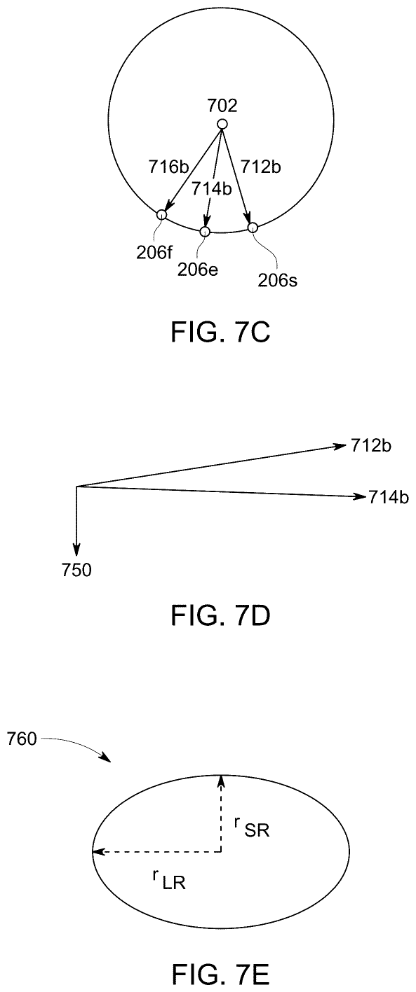

At step 430, a radius of the overlapping loop is calculated based on a detected overlap between respective electrodes. For example, the distances between each of the plurality of electrodes 206a-206u (uncoiled longitudinal distance) is stored within the memory 116 of the processor 140. Based on which of the plurality of electrodes 206a-206u overlap each other and/or which of the plurality of electrodes 206a-206u overlap each other, the circumference of the overlapping loop is calculated. For instance, if the proximal-most electrode 206a overlaps the distal-most electrode 206u, and none of the other electrodes 206b-206t overlap each other, the circumference of the overlapping loop is approximately the distance (uncoiled longitudinal distance) between the proximal-most electrode 206a and the distal-most electrode 206u. The radius of the overlapping loop is determined based on the calculated circumference. In some embodiments, the methods and/or concepts illustrated in FIGS. 6A-7 are used to calculate the radius of the overlapping loop.

At step 440, the raw impedance-based locations of each of the plurality of electrodes 206a-206u are corrected based on the calculated radius of the overlapping loop. For instance, the raw impedance-based locations of each of the plurality of electrodes 206a-206u are fit to an overlapping loop having the calculated radius (see e.g., the transformation between the visualized distal feature 304 and the corrected distal feature 354 in FIG. 3).

In some embodiments, correcting the raw impedance-based positions of each electrode 206a-206u based on the calculated radius of the overlapping loop includes applying a radial scaling function to undeform the loop dimensions. There are a number of radial scaling functions, including for instance, a non-uniform scaling transformation (correcting the raw impedance-based positions into a circle), a uniform scaling transformation (maintaining existing elliptical features), a uniform scaling transformation with bounds (determining proportional scaling of each raw impedance-based electrode position from the hoop centroid), and/or other radial scaling transformations known in the art.

In some embodiments, the radial scaling function includes identifying a long-axis radius of the raw impedance-based electrode locations. The electrode farthest from a hoop centroid is the long-axis radius and an electrode perpendicular (or closest to perpendicular) with the long-axis radius is the short axis radius (see e.g., FIG. 7E). If the long-axis radius is different from the short axis radius, the overlapping loop is elliptical, according to some embodiments. The non-uniform scaling transformation transforms both the long-axis radius (r_{LR}) and the short axis radius (r_{SR}) to the calculated radius (r_{calculated}) from step 430. In other words, r_{LR}=r_{SR}=r_{calculated}. The uniform scaling transformation determines a corrected short radius

$${r}_{correctedSR} = \frac{2 {r}_{calculated}}{\frac{{r}_{LR}}{{r}_{SR}} + 1}$$

and a corrected long-radius r_{correctedLR}=(r_{LR}/r_{SR})r_{correctedSR}. The uniform radial transformation with bounds determines a corrected short radius

$${r}_{correctedSR} = \frac{2 {r}_{calculated}}{min ( \frac{{r}_{LR}}{{r}_{SR}} , thresh ) + 1}$$

and a corrected long-rauius

$${r}_{correctedLR} = min ( \frac{{r}_{LR}}{{r}_{SR}} , thresh ) {r}_{correctedSR} ,$$

where a bound (thresh) on the ratio of measured long and short axis radii is provided.

Based on the corrected impedance-based locations of the plurality of electrodes, a corrected visualized catheter may be displayed via display device 144.

FIG. 5 is a flow chart of an exemplary method 420 of determining whether an overlapping loop is formed by the distal feature, according to some embodiments. At step 510, a best fit plane is calculated based on the raw impedance-based electrode positions. The best fit plane is a two-dimensional plane oriented substantially orthogonal to a central winding axis of the distal feature 204. The central winding axis of the distal feature 204 intersects the best fit plane at the centroid of the loop.

At step 520, the raw impedance-based positions of each of the plurality of electrodes 206a-206u are projected on the best fit plane. The best fit plane is two-dimensional, so the axial position of each of the plurality of electrodes 206a-206u is removed—only the (x, y) coordinates of each of the plurality of electrodes 206a-206u (i.e., the position relative to the central winding axis) is projected onto the best fit plane, according to some embodiments.

At step 530, vectors are generated from the centroid of the loop to each of the measured raw impedance-based positions of the plurality of electrodes 206a-206u projected onto the best fit plane. For example, a vector is generated from the centroid of the loop to the proximal-most electrode 206a (or the measured impedance-based position of the proximal-most electrode) on the best fit plane.

At step 540, a cross product of a first generated vector crossed with a second generated vector is calculated. For instance, a first generated vector from the centroid to the measured impedance-based position of the proximal-most electrode is crossed with a second generated vector from the centroid to the measured impedance-based position of the distal-most electrode.

At step 550, a determination is made as to whether the calculated cross product is positive or negative. If the calculated cross product is negative, the electrode pair in-question (i.e., using the example above, the proximal-most electrode 206a and the distal-most electrode 206u) does not overlap. If the calculated cross product is positive, a determination is made that the electrode pair in-question overlaps. In some embodiments, the method 420 repeats steps 540 and 550 for a plurality of different electrode pairs to identify whether an overlapping loop is formed. If an overlapping loop is determined to be formed by the distal feature, the method 420 proceeds to step 430.

FIG. 6 is a flow chart of an exemplary method 430 of determining a radius of an overlapping loop, according to some embodiments. At step 610, data of the distances (uncoiled longitudinal distance) between electrode pairs is stored. In some embodiments, the data is stored in the memory 140 of the processor 116.

At step 620, the shortest distance between overlapping electrode pairs is identified. For example, if the distal-most electrode 206u overlaps the proximal-most electrode 206a and the electrode 206b (and no other electrode overlap), the distance between the distal-most electrode 206u and the electrode 206b is the shortest overlapping distance. In other examples, various other overlapping electrode pairs may be compared to determine the shortest distance between all overlapping electrode pairs. In some embodiments, the shortest distance between overlapping electrode pairs is approximately equal to the circumference of the overlapping loop. In some embodiments, a plurality of overlapping electrode pairs are identified and the distances (uncoiled longitudinal distance) between the identified electrode pairs are compared to each other to determine the shortest distance between overlapping electrode pairs.

At step 630, a dot product between the overlapping electrode pair with the shortest distance therebetween is calculated. For instance, if the shortest distance between overlapping electrode pairs is identified as the distal-most electrode 206u and the electrode 206b, the dot product of the centroid to the measured impedance-based position of the distal-most electrode 206u is dotted with a second generated vector from the centroid to the measured impedance-based position of the electrode 206b. The dot product is indicative of the magnitude of the overlap, or in other words, by how much the electrode 206b overlaps the distal-most electrode 206u. In some embodiments, the magnitude of the overlap is added to the distance between overlapping electrode pairs to provide a more accurate circumference of the overlapping loop (as opposed to relying solely on the shortest distance between overlapping electrode pairs).

At step 640, the radius of the overlapping loop is determined based on the distances between overlapping electrode pairs and/or the dot product of overlapping electrode pair vectors. For example, the shortest distance between overlapping electrode pairs is approximately equal to the circumference of the overlapping loop. The magnitude of the overlap is determined via the dot product of the overlapping electrode pair vectors, and in some embodiments, is added to the shortest distance between overlapping electrode pairs to approximate the circumference of the overlapping loop. The radius of the overlapping loop is calculated from the determined circumference of the overlapping loop.

FIG. 7A is an exemplary coordinate system 700 of electrode position measurements of a variable loop catheter, according to some embodiments. The electrodes 206a-206u are wound around a central winding axis 720. The central winding axis 720 extends along the axial direction and runs through the centroid 702 of the loop. It should be noted that the best fit plane, as discussed above in refence to FIGS. 4-6, is a two-dimensional representation of the exemplary coordinate system 700 with the axial dimension removed. A first vector 712a is generated which extends from the centroid 702 (and/or from the central winding axis 720) to an electrode 206s. A second vector 714a extends from the centroid 702 (and/or from the central winding axis 720) to an electrode 206e, a third vector 716a extends from the centroid 702 to an electrode 206f, and a fourth vector 718a extends from the centroid 702 to an electrode 206g.

In some embodiments, including for example, the step 540 as shown and described in FIG. 5, the first vector 712a is crossed with one or more of the second vector 714a, the third vector 716a, and/or the fourth vector 718a. The direction of the resultant vector is based on the right-hand rule, with the direction being ‘negative’ if the electrodes are non-overlapping and ‘positive’ if the electrodes are overlapping (or vice versa, depending on orientation). In the example shown in FIG. 7A, 7B, the cross product of the first vector 712a crossed with the fourth vector 718a is negative, as the electrode 206s does not overlap with the electrode 206g. The cross product of the first vector 712a crossed with the third vector 716a is negative, as the electrode 206s does not overlap with the electrode 206f. The cross product of the first vector 712a crossed with the second vector 714a is positive, as the electrode 206s does overlap with the electrode 206e. Thus, the circumference of the loop in the exemplary coordinate system 700 is greater than the distance (uncoiled longitudinal distance) between the electrodes 206s and 206f and less than the distance (uncoiled longitudinal distance) between the electrodes 206s and 206c.

In some embodiments, the circumference of the loop is approximated by determining which of the electrodes the distal-most electrode 206u overlaps. For example, in the exemplary coordinate system 700, the distal-most electrode 206u overlaps with electrodes 206g, 206f, 206c, 206d, 206c, 206b, and 206a. The shortest distance between the overlapping electrode pairs is the distance (uncoiled longitudinal distance) between electrodes 206u and 206g, and therefore, the circumference of the loop is approximately the distance between electrodes 206u and 206g. In some embodiments, the circumference of the loop may also take into account the magnitude of overlap between electrodes 206u and 206g (in this example). As described above with respect to FIG. 6, in some embodiments the dot product of the two vectors associated with the overlapping electrodes (e.g., electrodes 206u and 206g in this example) provide an indication of the magnitude of overlap between the electrodes. In some embodiments, the magnitude of the overlap may be added to the circumference calculated based on the distance between the respective electrodes 206u and 206g.

FIG. 7B is an exemplary coordinate system 710 of electrode position measurements of a variable loop catheter, according to some embodiments. The circumference of the loop in the exemplary coordinate system 710 is greater than the loop of the exemplary coordinate system 700. For instance, the electrode 206s does not overlap with the electrode 206c (i.e., the cross product of first vector 712b crossed the second vector 714b is negative). Third vector 716b is also shown. Likewise, the distal-most electrode 206u does not overlap with the electrode 206g. In the exemplary coordinate system 710, the distal-most electrode 206u overlaps with electrodes 206f, 206c, 206d, 206c, 206b, and 206a. The shortest distance between the overlapping electrode pairs is the distance (uncoiled longitudinal distance) between electrodes 206u and 206f, and therefore, the circumference of the loop is approximately the distance between electrodes 206u and 206f.

FIG. 7C is a top view of the exemplary coordinate system 710 as shown in FIG. 7B, with the electrodes 206a-d, 206g-r, and 206u removed for viewing purposes, according to some embodiments. The first vector 712b does not overlap with either the second vector 714b or the third vector 716b. FIG. 7D is a view of the cross product 750 between the second vector 714b crossed the first vector 712b on the best fit plane. The cross product 750 is negative, indicating there is no electrode overlap. It should be noted that the cross product is dependent on which vector is crossed first—so to determine electrode overlap, the proximal electrode should be crossed with the distal electrode (or vice-versa, and the positive/negative relationship will swap). FIG. 7E is a diagrammatic view of an exemplary elliptical catheter loop 760, according to some embodiments. The radial scaling function described in FIG. 4 may identify the long-axis radius (TLR) and the short axis radius (SR) and scale the raw impedance-based electrode locations accordingly.

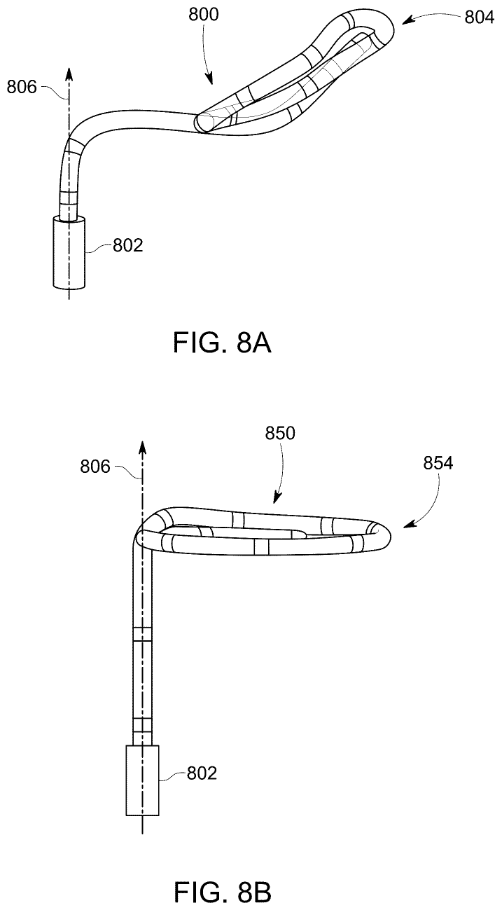

FIG. 8A is a visualized display of raw impedance-based catheter position, according to some embodiments. A visualized catheter 800 includes a visualized shaft 802 extending along an axial shaft axis 806, and a visualized distal feature 804. The visualized distal feature 804 is positioned relative to the axial shaft axis 806 in an irregular orientation. In other words, the physical constraints/dimensions of the catheter would not allow the distal feature to be positioned in the manner displayed by the visualized catheter 800, and therefore, a distortion has occurred. The raw impedance-based catheter position measurements may be distorted by non-homogenous conductive properties of different biologic tissue within a patient's body. For example, muscular tissue, vascular tissue, bone, cartilage, blood, etc., have different conductive properties which could create a non-homogeneous impedance field. The non-homogeneous impedance field can distort the raw impedance-based catheter position measurements, resulting in an imperfect, irregular shape of the visualized distal feature 804 relative to the axial shaft axis 806. In some embodiments, the catheter includes one or more shaft sensors located on the catheter shaft that provide position and orientation measurements with a high degree of confidence (relative to the raw impedance-based position data of the plurality of electrodes 206a-206u). The one or more shaft sensors are utilized to generate the axial shaft axis 806, and thus, it may be beneficial to correct the raw impedance-based position data of the plurality of electrodes based on the measured position of the catheter shaft and the physical constraints of the catheter.

FIG. 8B is a visualization display of corrected impedance-based catheter position, according to some embodiments. A corrected catheter 850 includes the visualized shaft 802 extending along the axial shaft axis 806, and a corrected distal feature 854. The corrected catheter 850 is generated based on raw impedance-based position data and corrected via the measured position of the catheter shaft and the physical constraints of the catheter.

FIGS. 9A-C are isometric front views of a loop catheter 200 with maximum deflection bounds, according to some embodiments. FIG. 9A is a front view of the catheter 200 with the proximal-most electrode 206a having a longitudinal range of deflection 900. The physical constraints (size, geometry, elasticity of materials, etc.) of the catheter 200 do not allow the proximal-most electrode 206a to deflect beyond the longitudinal range of deflection 900 relative to the catheter shaft 202. Therefore, if the measured raw impedance-based position of the proximal-most electrode 206a is outside of the longitudinal range of deflection 900 relative to the catheter shaft 202 and/or the catheter shaft axis 806, then a correction to the raw impedance-based positions may be necessary.

FIG. 9B is a front view of the catheter 200 in a maximum deflection state, according to some embodiments. The maximum deflection state may occur, for example, if the distal feature 204 is caught against tissue as the catheter 200 is inserted through the patient vasculature. The proximal-most electrode 206a is pulled to a minimum axial bound in the maximum deflection state illustrated in FIG. 9B. FIG. 9C is a front view of the catheter 200 in a second maximum deflection state, according to some embodiments. The second maximum deflection state may occur, for example, if the distal feature 204 is caught against tissue as the catheter 200 is inserted through the patient vasculature. The proximal-most electrode 206a is pulled to a maximum axial bound in the second maximum deflection state illustrated in FIG. 9C. The minimum axial bound and the maximum axial bound form the axial range of deflection 902 of the proximal-most electrode. If the measured raw impedance-based position of the proximal-most electrode 206a is outside of the axial range of deflection 902 relative to the catheter shaft 202 and/or the catheter shaft axis 806, then a correction to the raw impedance-based positions may be necessary.

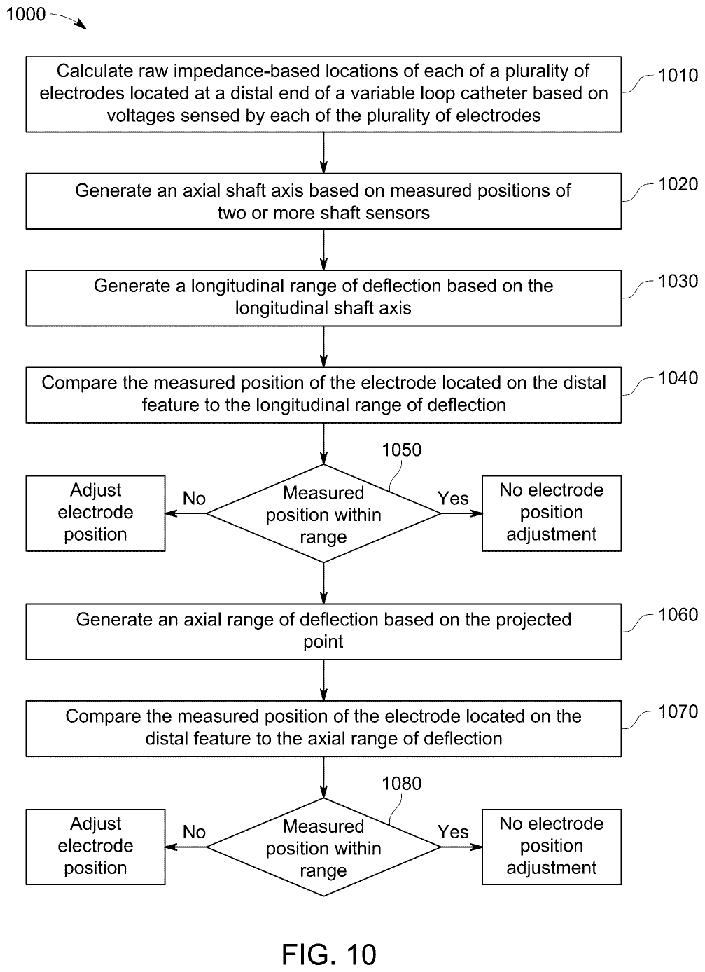

FIG. 10 is a flow chart of a method 1000 of rectifying impedance-based electrode positions for electrodes on a distal feature of a catheter, according to some embodiments. At step 1010, the raw impedance-based locations of each of a plurality of electrodes 206a-206u located at a distal end of a variable loop catheter 200 are calculated based on voltages sensed by each of the plurality of electrodes 206a-206u.

At step 1020 an axial shaft axis is generated based on measured positions of two or more shaft sensors. In some embodiments, the two or more shaft sensors include magnetic-based sensors, e.g., a coil configured to sense a generated magnetic field. In some embodiments, the two or more shaft sensors include impedance-based sensors or electrodes. The impedance-based sensors/electrodes have a larger surface area than the plurality of electrodes 206a-206u on the distal feature 204, and therefore may have higher sensitivity and more accuracy than the plurality of electrodes 206a-206u on the distal feature 204.

At step 1030 a longitudinal range of deflection is generated based on the axial shaft axis 806 and/or the physical constraints of the catheter 200. In some embodiments, the physical constraints of the catheter 200 are stored in the memory 140 of the processor 116. The physical constraints of the catheter 200 include a maximum longitudinal deflection of one or more of the plurality of electrodes 206a-206u relative to the catheter shaft 202, including for example, the longitudinal range of deflection 900 shown in FIG. 9A.

At step 1040 the raw impedance-based location(s) of the electrode(s) are compared to the longitudinal range of deflection. For example, the raw impedance-based location of the proximal-most electrode 206a is compared to the longitudinal range of deflection 900 to determine whether the raw impedance-based location of the proximal-most electrode 206a is within the longitudinal range of deflection 900. At step 1050, a determination is made as to whether the raw impedance-based location(s) of the electrode(s) are within the longitudinal range of deflection. If the raw impedance-based location(s) of the electrode(s) are outside of the longitudinal range of deflection, the measured electrode position is corrected, i.e., moved within the longitudinal range of deflection. If the raw impedance-based location(s) of the electrode(s) are within of the longitudinal range of deflection, no electrode position correction may be required.

At step 1060 an axial range of deflection generated based on the axial shaft axis 806 and/or the physical constraints of the catheter 200. In some embodiments, the physical constraints of the catheter 200 are stored in the memory 140 of the processor 116. The physical constraints of the catheter 200 include a maximum axial deflection and a minimum axial deflection of one or more of the plurality of electrodes 206a-206u relative to the catheter shaft 202, including for example, the longitudinal range of deflection 902 shown in FIGS. 9B-C.

At step 1070 the raw impedance-based location(s) of the electrode(s) are compared to the axial range of deflection. For example, the raw impedance-based location of the proximal-most electrode 206a is compared to the axial range of deflection 902 to determine whether the raw impedance-based location of the proximal-most electrode 206a is within the axial range of deflection 900. At step 1080, a determination is made as to whether the raw impedance-based location(s) of the electrode(s) are within the axial range of deflection. If the raw impedance-based location(s) of the electrode(s) are outside of the axial range of deflection, the measured electrode position is corrected, i.e., moved within the axial range of deflection. If the raw impedance-based location(s) of the electrode(s) are within of the axial range of deflection, no electrode position correction may be required.

For completeness, the methods described herein may be methods that are embedded within a set of human or machine-readable instructions that are comprised within a computer-readable medium or record carrier, or that are comprised within a computer program product. The instructions are such that, when executed by a computer or processor, such as the processor within the medical positioning system as described herein or a processor of a general-purpose computer system, the computer or processor causes the system to perform the methods described herein.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### Numbered Clauses

1. A method of displaying a position of a variable loop catheter that includes a distal feature having a plurality of electrodes located along a length of the distal feature, the method comprising:
calculating raw impedance-based positions of each of the plurality of electrodes located at a distal end of the variable loop catheter based on voltages sensed by each of the plurality of electrodes;
determining whether a loop is formed by the distal feature based on the raw impedance-based positions of each of the plurality of electrodes;
calculating a radius of the loop formed by the distal feature based on a detected overlap between respective electrodes; and correcting the measured raw impedance-based positions of each of the plurality of electrodes based on the calculated radius of the loop.

2. The method of clause 1, further comprising:
generating a best fit plane for the loop;
projecting the measured raw impedance-based positions of each of the plurality of electrodes on the best fit plane;
calculating a residual of the measured raw impedance-based positions on the best fit plane;
comparing the calculated residual to a threshold.

3. The method of clause 1 or clause 2, further comprising:
generating a vector from a centroid of the loop to the measured raw impedance-based positions of each of the plurality of electrodes;
calculating a cross product of a first generated vector crossed a second generated vector, wherein a positive cross product is indicative of an overlapping electrode pair.

4. The method of any of clauses 1 to 3, further comprising:
storing data of circumferential distances between one or more electrode pairs;
identifying one or more overlapping electrode pairs;
identifying a shortest circumferential distance between the one or more overlapping electrode pairs; and
determining a circumference of the loop based on the shortest circumferential distance between the one or more overlapping electrode pairs.

5. The method of clause 4, further comprising:
generating a vector from a centroid of the loop to the measured raw impedance-based positions of each of the plurality of electrodes;
calculating a dot product of a first generated vector dotted a second generated vector, wherein the first generated vector and the second generated vector correspond to the overlapping electrode pair with the shortest circumferential distance therebetween;
determining a magnitude of overlap based on the calculated dot product.

6. The method of any of clauses 1 to 5, wherein correcting the measured raw impedance-based positions of each of the plurality of electrodes based on the determined radius of the loop includes applying a radial scaling function to undeform the measured raw impedance-based positions of each of the plurality of electrodes.

7. The method of clause 6, wherein the radial scaling function is applied to a plane of projected electrode locations.

8. A medical positioning system, comprising:
a variable loop catheter including an elongate shaft and a distal feature including a plurality of distal electrodes, wherein the plurality of distal electrodes sense an impedance field; and
an electronic control unit (ECU) in communication with the catheter, the ECU receiving the sensed impedance field at the plurality of distal electrodes to measure a raw impedance-based position of each of the plurality of distal electrodes,
wherein the ECU determines whether the distal feature forms a loop in an overlapping state based on the measured raw impedance-based positions of each of the plurality of distal electrodes, and
wherein the ECU corrects the measured raw impedance-based positions of each of the plurality of distal electrodes based on detected characteristics of the loop in the overlapping state.

9. The medical positioning system of clause 8, wherein the detected characteristics of the loop in the overlapping state include a radius of the loop.

10. The medical positioning system of clause 9, wherein the ECU calculates the radius of the loop in the overlapping state by identifying an overlapping electrode pair and referencing a circumferential distance between the overlapping electrode pair.

11. The medical positioning system of clause 10, wherein the ECU identifies the overlapping electrode pair by translating the measured raw impedance-based positions of the plurality of distal electrodes onto a coordinate system and generating vectors from a centroid of the coordinate system to each of the measured raw impedance-based positions of the plurality of distal electrodes.

12. The medical positioning system of clause 11, wherein a first vector is crossed with a second vector to determine whether a first electrode overlaps a second electrode.

13. The medical positioning system of any of clauses 8 to 12, wherein the ECU identifies a long-axis radius of the distal feature and a short-axis radius of the distal feature, wherein the ECU applies a radial scaling function to the measured raw impedance-based positions.

14. The medical positioning system of clause 13, wherein the radial scaling function includes one or more of a non-uniform scaling transformation, a uniform scaling transformation, and a uniform scaling transformation with bounds.

15. The medical positioning system of any of clauses 8 to 14, wherein the variable loop catheter includes one or more electromagnetic sensors located on the elongate shaft, wherein the one or more electromagnetic sensors communicate measure a shaft position and orientation.

16. The medical positioning system of clause 15, wherein the ECU generates a longitudinal range of deflection based on the shaft position and generates an axial range of deflection based on the shaft position, wherein the ECU corrects the measured raw impedance-based positions of each of the plurality of electrodes in an longitudinal direction if the measured raw impedance-based positions are outside of a longitudinal range of deflection, and wherein the ECU corrects the measured raw impedance-based positions of each of the plurality of electrodes in an axial direction if the measured raw impedance-based positions are outside of the axial range of deflection.

17. A method of correcting impedance-based electrode positions for electrodes on a distal feature of a catheter, the method comprising:
calculating raw impedance-based positions of each of a plurality of electrodes located at a distal end of a variable loop catheter based on voltages sensed by each of the plurality of electrodes;
generating a longitudinal shaft axis between two or more shaft sensors;
generating a longitudinal range of deflection based on the longitudinal shaft axis;
comparing the measured raw impedance-based positions of each of the plurality of electrodes to the longitudinal range of deflection; and
correcting the measured raw impedance-based positions of each of the plurality of electrodes in a longitudinal direction if the measured raw impedance-based positions are outside of the longitudinal range of deflection.

18. The method of clause 17, further comprising:
generating an axial range of deflection based on the longitudinal shaft axis;
comparing the measured raw impedance-based positions of each of the plurality of electrodes to the axial range of deflection; and
correcting the measured raw impedance-based positions of each of the plurality of electrodes in an axial direction if the measured raw impedance-based positions are outside of the axial range of deflection.

19. The method of clause 17 or clause 18, further comprising:
determining whether a loop is formed by the distal feature based on the raw impedance-based locations of each of the plurality of electrodes;
calculating a radius of the loop formed by the distal feature based on a detected overlap between respective electrodes; and
correcting the measured raw impedance-based positions of each of the plurality of electrodes based on the calculated radius of the loop.

20. The method of clause 19, wherein correcting the measured raw impedance-based positions of each of the plurality of electrodes based on the calculated radius of the loop includes applying a radial scaling function to undeform the measured positions of each of the plurality of electrodes.

What is claimed is:

1. A method of displaying a position of a variable loop catheter that includes a distal feature having a plurality of electrodes located along a length of the distal feature, the method comprising:
calculating raw impedance-based positions of each of the plurality of electrodes located at a distal end of the variable loop catheter based on voltages sensed by each of the plurality of electrodes;
determining whether a loop is formed by the distal feature based on the raw impedance-based positions of each of the plurality of electrodes;
calculating a radius of the loop formed by the distal feature based on a detected overlap between respective electrodes; and
correcting the measured raw impedance-based positions of each of the plurality of electrodes based on the calculated radius of the loop.

2. The method of claim 1, further comprising:
generating a best fit plane for the loop;
projecting the measured raw impedance-based positions of each of the plurality of electrodes on the best fit plane;
calculating a residual of the measured raw impedance-based positions on the best fit plane;
comparing the calculated residual to a threshold.

3. The method of claim 1, further comprising:
generating a vector from a centroid of the loop to the measured raw impedance-based positions of each of the plurality of electrodes;
calculating a cross product of a first generated vector crossed a second generated vector,
wherein a positive cross product is indicative of an overlapping electrode pair.

4. The method of claim 1, further comprising:
storing data of circumferential distances between one or more electrode pairs;
identifying one or more overlapping electrode pairs;
identifying a shortest circumferential distance between the one or more overlapping electrode pairs; and
determining a circumference of the loop based on the shortest circumferential distance between the one or more overlapping electrode pairs.

5. The method of claim 4, further comprising:
generating a vector from a centroid of the loop to the measured raw impedance-based positions of each of the plurality of electrodes;
calculating a dot product of a first generated vector dotted a second generated vector, wherein the first generated vector and the second generated vector correspond to the overlapping electrode pair with the shortest circumferential distance therebetween;
determining a magnitude of overlap based on the calculated dot product.

6. The method of claim 1, wherein correcting the measured raw impedance-based positions of each of the plurality of electrodes based on the determined radius of the loop includes applying a radial scaling function to undeform the measured raw impedance-based positions of each of the plurality of electrodes.

7. The method of claim 6, wherein the radial scaling function is applied to a plane of projected electrode locations.

8. A medical positioning system, comprising:
a variable loop catheter including an elongate shaft and a distal feature including a plurality of distal electrodes, wherein the plurality of distal electrodes sense an impedance field; and
an electronic control unit (ECU) in communication with the catheter, the ECU receiving the sensed impedance field at the plurality of distal electrodes to measure a raw impedance-based position of each of the plurality of distal electrodes,
wherein the ECU determines whether the distal feature forms a loop in an overlapping state based on the measured raw impedance-based positions of each of the plurality of distal electrodes, and
wherein the ECU corrects the measured raw impedance-based positions of each of the plurality of distal electrodes based on detected characteristics of the loop in the overlapping state.

9. The medical positioning system of claim 8, wherein the detected characteristics of the loop in the overlapping state include a radius of the loop.

10. The medical positioning system of claim 9, wherein the ECU calculates the radius of the loop in the overlapping state by identifying an overlapping electrode pair and referencing a circumferential distance between the overlapping electrode pair.

11. The medical positioning system of claim 10, wherein the ECU identifies the overlapping electrode pair by translating the measured raw impedance-based positions of the plurality of distal electrodes onto a coordinate system and generating vectors from a centroid of the coordinate system to each of the measured raw impedance-based positions of the plurality of distal electrodes.

12. The medical positioning system of claim 11, wherein a first vector is crossed with a second vector to determine whether a first electrode overlaps a second electrode.

13. The medical positioning system of claim 8, wherein the ECU identifies a long-axis radius of the distal feature and a short-axis radius of the distal feature, wherein the ECU applies a radial scaling function to the measured raw impedance-based positions.

14. The medical positioning system of claim 13, wherein the radial scaling function includes one or more of a non-uniform scaling transformation, a uniform scaling transformation, and a uniform scaling transformation with bounds.

15. The medical positioning system of claim 8, wherein the variable loop catheter includes one or more electromagnetic sensors located on the elongate shaft, wherein the one or more electromagnetic sensors communicate measure a shaft position and orientation.

16. The medical positioning system of claim 15, wherein the ECU generates a longitudinal range of deflection based on the shaft position and generates an axial range of deflection based on the shaft position, wherein the ECU corrects the measured raw impedance-based positions of each of the plurality of electrodes in an longitudinal direction if the measured raw impedance-based positions are outside of a longitudinal range of deflection, and wherein the ECU corrects the measured raw impedance-based positions of each of the plurality of electrodes in an axial direction if the measured raw impedance-based positions are outside of the axial range of deflection.

17. A method of correcting impedance-based electrode positions for electrodes on a distal feature of a catheter, the method comprising:
calculating raw impedance-based positions of each of a plurality of electrodes located at a distal end of a variable loop catheter based on voltages sensed by each of the plurality of electrodes;
generating a longitudinal shaft axis between two or more shaft sensors;
generating a longitudinal range of deflection based on the longitudinal shaft axis;
comparing the measured raw impedance-based positions of each of the plurality of electrodes to the longitudinal range of deflection; and
correcting the measured raw impedance-based positions of each of the plurality of electrodes in a longitudinal direction if the measured raw impedance-based positions are outside of the longitudinal range of deflection.

18. The method of claim 17, further comprising:
generating an axial range of deflection based on the longitudinal shaft axis;
comparing the measured raw impedance-based positions of each of the plurality of electrodes to the axial range of deflection; and
correcting the measured raw impedance-based positions of each of the plurality of electrodes in an axial direction if the measured raw impedance-based positions are outside of the axial range of deflection.

19. The method of claim 18, further comprising:
determining whether a loop is formed by the distal feature based on the raw impedance-based locations of each of the plurality of electrodes;
calculating a radius of the loop formed by the distal feature based on a detected overlap between respective electrodes; and
correcting the measured raw impedance-based positions of each of the plurality of electrodes based on the calculated radius of the loop.

20. The method of claim 19, wherein correcting the measured raw impedance-based positions of each of the plurality of electrodes based on the calculated radius of the loop includes applying a radial scaling function to undeform the measured positions of each of the plurality of electrodes.
